(19) United States
(12) Patent Application Publication
(10) Pub. No.: US 2015/0079027 A1
(43) Pub. Date: Mar. 19, 2015

(54) ANTITUMOR PROTOCOL

(71) Applicant: DENDRITHERAPEUTICS, INC., Danville, CA (US)

(72) Inventor: Anthony E. Maida, III, Danville, CA (US)

(73) Assignee: DENDRITHERAPEUTICS, INC., Danville, CA (US)

(21) Appl. No.: 14/386,786

(22) PCT Filed: Mar. 26, 2013
(86) PCT No.: PCT/US13/33892

§ 371 Date: Sep. 19, 2014

Related U.S. Application Data
(60) Provisional application No. 61/616,395, filed on Mar. 27, 2012.

(51) Int. Cl. A61K 39/00; A61N 5/10; A61K 38/20; A61K 39/395; C07K 16/28; A61K 38/45

(52) U.S. Cl. CPC A61K 39/0011; C07K 16/2818; A61K 38/45; A61K 38/2013; C12Y 204/02036; A61K 39/3955; A61N 5/10; C07K 2317/21; C07K 2319/55
USPC 424/85.2; 600/1

(57) ABSTRACT

Improved protocols that enhance the effect of immunotherapy against tumors are described. The protocol involves enhancing the immune response to an antitumor vaccine by maximizing the ability of T cells induced by the vaccine to extravasate into the tumor and also to modulate the effect of Tregs so that the effectiveness of the vaccine is not undermined. The former effect is achieved by radiation at the site of the tumor or lesions. This latter effect is achieved by an initial depletion of Tregs in combination with modulating their effect during the vaccine administration protocol.

### RELATED APPLICATION

This application claims benefit of U.S. application Ser. No. 61/616,395 filed 27 Mar. 2012. The entire contents of this document are incorporated herein by reference.

### TECHNICAL FIELD

The invention is in the field of antitumor therapy. In particular, it concerns improved protocols that enhance the effectiveness of immunotherapy against tumors, including solid tumors.

### BACKGROUND ART

It has long been an approach to antitumor therapy to administer vaccines that are designed to induce an immune response against the tumor—both humoral and T cell mediated responses. Various vaccines have been proposed, including those where the active ingredient is one or more peptides or other antigen moieties which are associated with the tumors—i.e., tumor-associated antigens (TAA). Often these are formulated as peptide or protein vaccines, although in some instances, peptide or protein antigens are generated in vivo after administration of nucleic acids that encode them. A large number of formulations of such vaccines, including liposomal preparations, nanoparticulate based formulations, and free drug formulations have been employed. Other antigen-providing vaccines include autogeneic or allogeneic tumor cells that display antigens similar to those of the tumor-associated antigens of the subject to be treated. Such vaccines are disclosed, for example, in U.S. Pat. No. 7,740,837 and U.S. Pat. No. 8,293,252. The latter patent describes universal vaccines that are effective in displaying antigens that represent tumors of a wide variety of types. A particularly effective multivalent vaccine is described in PCT publication WO2005/037190, incorporated herein by reference. These vaccines comprise multiple antigens and thereby enhance the effectiveness of the vaccine itself.

Various approaches have been employed to attempt to enhance the effectiveness of the immune response to tumors. One approach, as exemplified by the two U.S. issued patents discussed above, is to modify the allogeneic or autogeneic tumor cells to decrease expression or activity of an immunosuppressant. In another approach, Curiel, T. J., et al., J. Clin. Invest. (2007) 117:1167-1174 suggests the use of denileukin diftitox (Ontak®) to deplete T regulating cells (Tregs) and to employ cytotoxic T-lymphocyte-associated antigen-4 (CTLA-4) as a blockade. These drugs deplete or block the function of Tregs, thus enhancing the ability of the immune system to reject the tumor. In addition, Vieweg, J., et al., Clin. Cancer Res. (2007) 13:(2 suppl.) 727s-732s report that administering Ontak® followed by vaccination with RNA transfected dendritic cells improved the stimulation of tumor-specific T cell responses in renal cell carcinoma patients as compared to vaccination alone.

It has been shown that radiation of tumors effects a proinflammation response and modulates expression of adhesion molecules in tumors. For example, Baluna, R. G., et al., Radiation Res. (2006) 166:819-831 reviewed the literature demonstrating that the expression of many adhesion molecules associated with vasculature is modulated by radiation. They propose that adhesion molecules are markers for radiation toxicity and markers for tumor response to radiation. Combination treatments of radiation with adhesion molecules are also proposed. Ganss, R., et al., Cancer Res. (2002) 162:1462-1470 report the effect of radiation to permit T cells to extravasate and thus to destroy the tumor, and that the combination of radiation with adoptive therapy administering T cells in combination with the radiation is effective in treating tumors. It has been reported that in one melanoma patient, the combination of radiation and the monoclonal antibody, ipilimumab, was able to induce a systemic antitumor response. New England Journal of Medicine (2012) 366:926-931.

It has also been demonstrated, for example, by Read, S., et al., J. Exp. Med. (2000) 192:295-302 that certain antibodies, such as antibodies against CTLA-4, are able to block the activity of Tregs as well as blocking suppression of activated T cells. Indeed, cancer regression and autoimmunity have been reported to be induced by CTLA-4 blockage in patients with metastatic melanoma by Phan, G. Q., et al., PNAS (2003) 100:8372-8377.

It has now been found that a protocol can be designed that takes advantage of the interplay of a multiplicity of factors so as to induce an effective immune response against tumors in vivo.

### DISCLOSURE OF THE INVENTION

The invention is directed to an improved protocol which is an immunomodulatory conditioning regimen for treating tumors in subjects, especially in those subjects bearing solid tumors or those with discernible lesions. The protocol involves enhancing the immune response to an antitumor vaccine by maximizing the ability of T cells induced by the vaccine to extravasate into the tumor and also to modulate the effect of Tregs so that the effectiveness of the vaccine is not undermined. The former effect is achieved by radiation at the site of the tumor or lesions. This latter effect is achieved by an initial depletion of Tregs in combination with modulating their effect during the vaccine administration protocol.

Thus, the invention is directed to a method to induce an immune response to a tumor in a subject which method comprises: initially depleting T regulatory cells (Tregs) in said subject, followed by irradiating especially a solid tumor or discrete lesion to modulate adhesion molecule expression; and administering an antitumor vaccine in tandem with modulating Tregs activity.

### MODES OF CARRYING OUT THE INVENTION

The invention protocol uses an immunomodulatory and conditioning regimen that will enhance both the induction and effector phases of the immune response, as well as, radiation induced upregulation of tumor neovascular adhesion molecules, combined with a cancer vaccine for the treatment of tumors such as melanoma.

It is known the efficacy of the induction phase can be improved by blocking the negative regulators of the activation of effector T cells (Korman, et al., (2005)). Cytotoxic T cell associated antigen-4 (CTLA-4) is expressed on activated T cells as a regulatory brake that halts T cell activation. Blocking the activity of CTLA-4 allows greater expansion of all T cell populations, presumably including those with anti-tumor activity.

Thus co-administration of binding agents for CTLA-4 allows preferential activation and more robust expansion of T cells that respond to tumor vaccine. Antibodies against CTLA-4 are being evaluated in clinical trials in melanoma and when used with a cancer vaccine have demonstrated meaningful clinical responses in approximately 20% of patients (Phan, G. Q., et al., (2003, supra); Weber, J., The Oncologist (2008) 13 (suppl.4):16-25) with the development of significant, but treatable autoimmunity in a subset of subjects. The development of autoimmunity has been associated with a more robust and durable clinical tumor response.

The effector phase of the immune response to a cancer vaccine can be enhanced by eliminating or decreasing immunosuppressive regulatory T cells before immunization. A molecule that targets the high affinity IL-2 receptor (CD25), which is expressed at high density on regulatory T cells, is denileukin diftitox (Ontak®, Eisai, Inc.). When administered to patients with melanoma, Ontak® depletes regulatory T cells and this has resulted in the production of melanoma specific CD8 positive T cells in approximately 90% of patients. (Mahnke, K., et al., Int. J. Cancer (2007) 120:2723-2733; Chesney, et al., ASCO Abstract (2006) 18010.)

The first step in the protocol is designed to deplete the level of Tregs circulating in the subject. Tregs are understood to be essential in preventing autoimmunity. These cells are CD4⁺/CD25⁺ and are known to suppress the function and proliferation of tumor-specific CD4⁺ and CD8⁺ effector T cells, and thus to inhibit adaptive and innate immune responses in vivo. As noted above, a known agent for depletion of these cells is denileukin diftitox, trademarked Ontak® which is a recombinant fusion protein that contains the catalytic and membrane translocation domain of diphtheria toxin fragments A and B (Met1-Thr387), wherein the binding domain for the diphtheria toxin receptor is replaced by the human IL-2 (Ala1-Thr133) thus targeting CD25-expressing cells. Other agents that specifically target CD25-expressing cells could also be used.

In one embodiment, the agent that depletes Tregs is administered before the vaccination regimen takes place. Generally, this administration is completed prior to the initiation of the vaccine or radiation protocol. Typically, the of the anti-CD25 binding agent is administered over a period of several weeks with each dose of intravenous or other parenteral administration occurring continuously over 2-8 days, typically 4-7 days or 5-6 days. More than one session or dose of administration of this Tregs-depleting agent can be employed—thus, 1, 2, 3 or 4 administrations may be used. Typically, these are spaced in order to avoid toxicity. The regimen for administration of the Tregs-depleting agent is thus performed as a precursor to the remainder of the protocol.

Before or during the vaccination protocol, the subjects are subjected to radiation directed at the tumor or, in some cases, to whole body irradiation. The effect of this radiation treatment is to induce remodeling of the vasculature so that extravasation of effector T cells into the tumor is enhanced. If the tumor to be treated is not a solid tumor or a tumor with defined lesions, this aspect of the protocol is optional and generally unnecessary. The effect of radiation is to ease the entry of the effector T cells elicited by the vaccine into solid tumors, so that the radiation can be administered immediately before or during the vaccination protocol. The level of radiation dosage will depend on whether the tumor is targeted directly or whole body radiation is employed and on the level of remodeling that needs to be effected. The radiation schedule can be integrated with the schedule for administration of the vaccine and with the schedule for the administration of anti-CTLA-4 antibody that modulates the effect of Tregs. Each of the radiation treatments may be scheduled at a time selected to correspond to a particular administration of the vaccine and/or the Tregs modulator.

In one embodiment, the radiation is conducted immediately preceding (e.g., about 12 hours-36 hours) the administration of the Tregs modulator. The parameters of the irradiation are designed to have the effect of enhancing an immune response, rather than directly treating the tumor itself.

The vaccination protocol itself employs any vaccine directed to eliciting an immune response to a tumor associated antigen. As noted above, the vaccine may be in the form of protein, nucleic acid, or autologous or allogeneic cells and may be univalent or multivalent. Techniques for administering antigens designed to elicit, in particular, a cellular response are well known. Typically, the administration of such vaccines is parenteral, typically intravenous. Depending on the vaccine chosen, the administration may be over a period of minutes, hours or days.

During the vaccination protocol, the function of Tregs is modulated according to the method of the invention, while the effectiveness of the effector T cells generated by the vaccine is unaffected. Thus, agents need to be chosen that are specific for Tregs as opposed to targeting effector T cells in general. One such agent that is particularly favored is anti-CTLA-4. Monoclonal antibodies that have this function are available, including tremelimumab which is an IgG1 human mAb and an alternative IgG1 human monoclonal mAb, ipilimumab. However, other agents that specifically target the function of Tregs while not substantially inhibiting antitumor T cells may be substituted. For example, any binding agent for CTLA-4 may be used, such as aptamers or other specific binding partners.

It should be noted that although mAb's are commercially available and convenient, mAb's per se would not be required. Clearly fragments of such antibodies, recombinantly produced forms, such as single-chain antibodies, antibody mimics, such as aptamers, and the various art-known modifications of traditional antibodies can be included. Thus, the CTLA-4 binding agent employed may include any of these functionalities. Anti-CTLA-4 antibodies thus include mimics, fragments, and various recombinantly produced or modified forms of native antibodies. If the vaccine includes allogeneic or autologous cells, these cells may be modified to produce the CTLA-4 binding agents as well.

These agents, too, are typically administered intravenously over a period of minutes, hours or days.

The administration of the “anti-Tregs function” agent and the vaccine is generally concurrent and/or tandem. It is within the scope of the invention to administer these compositions substantially or partially simultaneously.

The subjects of the enhanced protocol are generally mammalian subjects such as humans, companion animals such as dogs and cats, livestock such as pigs, cows and sheep, or laboratory animals for the purpose of optimizing the protocol. Thus, one aspect of the invention is to employ the protocol in laboratory-designed experiments utilizing non-human primates, dogs, mice, rats, rabbits or other common models to optimize dosages, determine safety and effectiveness, and design suitable formulations.

Types of cancers subject to this treatment include, but are not limited to, cancers of the colon, breast, lung, prostate, pancreas, liver, testicles, brain, kidney, endometrium, cervix, ovary, thyroid, or other glandular tissue, as well as squamous, melanoma, central nervous system, and carcinoma generally.

The following examples are intended to illustrate, not to limit the invention.

### EXAMPLE 1

### Phase I/II Clinical Study

Fifteen patients with stage III/IV metastatic melanoma are enrolled in the study. They are selected as having unresectable, measurable disease. They are selected as follows:

Eighteen (18) years old or older, with histologically confirmed unresectable stage III-IV melanoma by the 2001 modified American Joint Commission on Cancer staging system, and disseminated disease with at least two measurable metastatic lesions (cutaneous, lymph node, head or viscera) documented at screening/baseline on magnetic resonance imaging (MRI) or computed tomography (CT) (with and without contrast) of neck, thorax, abdomen, and pelvis within four weeks of onset of therapy. Also there must be evidence of active cerebral metastasis(es) on screen/baseline brain MRI/CT, and ECOG PS up to 2. They are excluded if they have active or untreated central nervous system metastasis, had been treated with immunosuppressive agents at the time of eligibility assessment, or had prior treatment with anti-CTLA-4 mAb, or with denileukin diftitox. They must not have history or clinical evidence of autoimmune disease or auto-antibodies (i.e., rheumatoid factor (RF), anti-nuclear antibodies). They must not have any prior malignancy from which they are disease free for less than 5 years with the exception of basal or squamous cell skin cancer, superficial bladder cancer, or in situ cervical carcinoma, or have any active infection. They are excluded if they exhibit chronic active Hepatitis B or C or are positive for Hepatitis C antibody or antibody to Hepatitis B surface antigen, or human immunodeficiency virus (HIV).

The protocol is as follows: This study is conducted over the course of two years. On each of day 0 and day 25, the subject is administered intravenously for 5 consecutive days with 9 μg/kg/day of denileukin diftitox (Ontak®). Following the administration of Ontak®, the modulation of Tregs by blocking CTLA-4 is maintained by administering a suitable monoclonal antibody that binds CTLA-4—either tremelimumab or ipilimumab at 10 mg/kg administered intravenously every 3 weeks for a total of 4 cycles followed by maintenance dosing of 10 mg/kg every 12 weeks. Thus, during the first year of the study, the antibody is administered at least at weeks 9, 12, 15, 27, 39 and 51 and in the second year at least at weeks 12, 24, 36 and 48.

In some patients, solid tumors or lesions are irradiated to modulate the immune response by increased expression of cell adhesion molecules on a tumor neovasculature, and by enhanced MHC-I expression and in an expanded peptide repertoire. The irradiation is not conducted for therapeutic purposes per se. In those patients where irradiation is performed, at least two sites are available so that a control site not being irradiated will be present.

Each target lesion or tumor is irradiated to a dose of 6 Gy in one lesion on days 41, 62, 83 and 104, 24 hours prior to each dose of the first 4 doses of anti-CTLA-4 to a maximum cumulative dose to any target lesion of 24 Gy. The dose is calculated at the isocenter for lesions treated using a three-dimensional, conformal technique. Lesions treated with an electron-beam technique will have the dose calculated at the isodose volume encompassing the lesion. Lesions treated with stereotactic techniques will be treated to the isodose volume encompassing the lesion, from 50% to 90%, at the discretion of the radiation oncologist.

The appropriate volume and technique will vary depending on the target lesion. In general, the volume (GTV) should encompass the lesion with expansions to CTV and PTV as appropriate to the technique used. No attempt is made to include surrounding normal tissues or draining lymph nodes. Expansions for differing techniques will necessarily differ, with the largest expansions for electron beam techniques and the smallest for stereotactic techniques. The actual expansions are at the discretion of the radiation oncologist. In all cases, it is not acceptable to treat a volume that creates a substantial risk of normal tissue toxicity. For example, treatment of a cluster of lung metastases in a way that creates a significant risk for radiation pneumonitis is unacceptable, as would be treatment of a cluster of hepatic metastases using a technique that would create a substantial risk for radiation hepatitis.

### RESULTS

Although in this phase I/II study, the vaccine itself is not administered, a beneficial effect on the cancer may be observed. Safety evaluation is done using conventional criteria and efficacy is based on assessment of immune response and tumor response.

Immune response and tumor measurements are conducted at screen/baseline, at weeks 6, 12, 27, and 51 during the first year and at weeks 24 and 48 during the second year, and at study termination.

In vitro immune response parameters include: IFN-gamma ELISA spot, ELISA to detect antibodies to tumor antigens, cytotoxicity assays to detect T cell killing of melanoma tumor cells, cytokine and adhesion molecule profiles, and gene expression profiles.

Tumor response is assessed by evaluation of measurable disease utilizing RECIST criteria. Measurable disease includes any lesion that can be accurately measured in at least one dimension (longest diameter to be recorded) as ≧20 mm with conventional techniques (X-ray, MRI, CT) or as ≧10 mm with spiral CT scan. All measurements are taken and recorded in metric notation (millimeters) using a ruler or calipers. Efficacy endpoints are defined as:
Complete Response (CR): Disappearance of all target lesions;
Partial Response (PR): At least a 30% decrease in the sum of the longest diameter of target lesions, taking as reference the baseline sum longest diameter;
Stable Disease (SD): Neither sufficient shrinkage to qualify for partial response nor sufficient increase to qualify for progressive disease, taking as reference the smallest sum longest diameter since the treatment started; and
Progressive Disease (PD), at least a 20% increase in the sum of the longest diameter of target lesions, taking as reference the smallest sum longest diameter recorded since the treatment started or the appearance of one or more new lesions.

All cutaneous lesions are serially photographed, digitally recorded, and analyzed by a software package that evaluates the appearance and dimensions of the lesions quantitatively (Photographic Imaging Management System). All MRI/CT images are similarly analyzed. These photographic and radiographic scanning images can then be evaluated by central reader/readers.

In addition, in the case of cutaneous lesions, biopsies are obtained, if possible, at baseline and following treatment for assessment of standard histology, immunocytochemistry for lymphocyte phenotype, and molecular pathology (i.e., cytokines, chemokines, and adhesion molecules).

Statistical analysis is applied to the results.

### EXAMPLE 2

### Clinical Efficacy Study

The protocol in Example 1 is followed except that a vaccine comprises tumor-associated antigens either per se or as exhibited on autologous or allogeneic cells is supplied intravenously in conjunction with the anti-CTLA-4 mAb. The vaccine itself may be administered at alternate weeks, concomitantly with the mAb, or in a schedule overlapping the administration of the mAb but independent of it.

1. A method to induce an immune response to a solid tumor or a tumor bearing lesions in a subject which method comprises the following steps:
a) initially depleting T regulatory cells (Tregs) in said subject, and after completion of step a) followed by
b) irradiating the tumor with radiation to modulate adhesion molecule expression to remodel the vasculature in said tumor; and
c) administering an antitumor vaccine along with modulating Tregs activity.

2. The method of claim 1 wherein said depleting Tregs is by administering to said subject an amount of denileukin diftitox (Ontak®) effective to deplete Tregs.

3. The method of claim 1 wherein said irradiating is of the whole body of the subject.

4. The method of claim 1 wherein said antitumor vaccine comprises allogeneic or autologous cells.

5. The method of claim 1 wherein said antitumor vaccine comprises a tumor-associated antigen or a fragment thereof.

6. The method of claim 1 wherein said antitumor vaccine comprises a nucleic acid for generating a tumor-associated antigen.

7. The method of claim 1 wherein the antitumor vaccine is a multivalent vaccine.

8. The method of claim 1 wherein modulating Tregs activity is by administering a binding agent for cytotoxic T-lymphocyte-associated antigen-4 (CTLA-4).

9. The method of claim 8 wherein the binding agent is an anti-CTLA-4 antibody or fragment thereof.

10. A method to induce an immune response to a tumor in a subject which method comprises:
a) initially depleting T regulatory cells (Tregs) in said subject, and after completion of step a) followed by
b) administering an antitumor vaccine along with modulating Tregs activity.

11. The method of claim 10 wherein said depleting Tregs is by administering to said subject an amount of denileukin diftitox (Ontak®) effective to deplete Tregs.

12. The method of claim 10 wherein said antitumor vaccine comprises allogeneic or autologous cells.

13. The method of claim 10 wherein said antitumor vaccine comprises a tumor-associated antigen or a fragment thereof.

14. The method of claim 10 wherein said antitumor vaccine comprises a nucleic acid for generating a tumor-associated antigen.

15. The method of claim 10 wherein the antitumor vaccine is a multivalent vaccine.

16. The method of claim 10 wherein modulating Tregs activity is by administering a binding agent for cytotoxic T-lymphocyte-associated antigen-4 (CTLA-4).

17. The method of claim 16 wherein the binding agent is an anti-CTLA-4 antibody, or fragment thereof.
